(19) United States
(12) Patent Application Publication
(10) Pub. No.: US 2025/0009936 A1
(43) Pub. Date: Jan. 9, 2025

(54) TISSUE REGENERATION WITH IN VITRO ACTIVATED EFFECTOR CELLS

(71) Applicant: Wake Forest University Health Sciences, Winston-Salem, NC (US)

(72) Inventors: Johanna BOLANDER, Winston-Salem, NC (US); Gustavo MOVIGLIA, Winston-Salem, NC (US); Anthony ATALA, Winston-Salem, NC (US)

(21) Appl. No.: 18/709,220

(22) PCT Filed: Nov. 15, 2022
(86) PCT No.: PCT/US2022/079864

§ 371 Date: May 10, 2024

Related U.S. Application Data
(60) Provisional application No. 63/279,316, filed on Nov. 15, 2021.

(51) Int. Cl. A61L 27/38; A61L 27/20; A61L 27/52; C12N 5/077

(52) U.S. Cl. CPC A61L 27/3834; A61L 27/20; A61L 27/52; C12N 5/0654; C12N 5/0655; A61L 2300/64; A61L 2430/02; A61L 2430/06; C12N 2500/84; C12N 2502/11; C12N 2502/1352; C12N 2533/80

(57) ABSTRACT

A composition comprising a co-culture of: a) activated peripheral blood mononuclear cells (PBMC); and b) stem or progenitor cells, is provided. The composition is useful for the regeneration of tissue in a subject in need thereof, and for forming of a tissue in vitro.

### RELATED APPLICATIONS

This application claims the benefit of U.S. Provisional Patent Application No. 63/279,316, filed Nov. 15, 2021, the contents of which are incorporated by reference herein.

### BACKGROUND

Knee trauma occurs primarily in the young population, with over 50% in individuals between the ages of 15 and 44. Post-trauma osteoarthritis (PTOA) begins early, even in the relative absence of symptoms. An outcomes study of PTOA demonstrated radiographic evidence of knee osteoarthritis (KOA) in 12-20% of patients after 5 years. Treatment for KOA involves a life-long combination of exercise, lifestyle modifications, corticosteroid injections, analgesics, and in severe cases, knee replacement. See Sharma, “Osteoarthritis of the Knee,” The New England Journal of Medicine, Jan. 7, 2021. Non-surgical treatment can only provide pain relief and does not slow down the progression of KOA.

Cell therapy with the administration of mesenchymal stem cells such as adipose-derived stromal cells has been studied for osteoarthritis treatment, and may have anti-inflammatory properties. However, although some studies showed a benefit from the treatment, some did not. See, e.g., van den Bosch, “Review: Osteoarthritis year in review 2020: biology,” Osteoarthritis and Cartilage 29 (2021) 143-150; Zhang et al., “Review: Progress in the use of mesenchymal stromal cells for osteoarthritis treatment,” Cytotherapy 23 (2021) 459-470; Soltani et al., “Safety and efficacy of allogenic placental mesenchymal stem cells for treating knee osteoarthritis: a pilot study,” Cytotherapy 21 (2019) 54-63. This treatment also only provides a short-term effect, mainly through pain reduction. Since little or no structural regeneration is achieved, the disease progresses after some time.

U.S. Patent Application Publication No. 2012/0171169 of Jaewoo Pak concerns compositions for treatment of bone diseases with adipose tissue-derived stem cells, platelet rich plasma, calcium chloride, and hyaluronic acid, and in some embodiments further comprising dexamethasone for treatment of cartilage diseases. However, there are no comparative examples indicating that this stem cell treatment yields an improved bone or cartilage cell recovery over other methods of stem cell administration known in the art.

Better treatment options for tissue regeneration such as for PTOA is needed.

### SUMMARY

Provided herein according to some embodiments is a composition comprising a co-culture of: a) activated peripheral blood mononuclear cells (PBMC); and b) stem or progenitor cells, wherein said activated PBMC and said stem or progenitor cells are present in a ratio of from 6:1, 5:1, 4.5:1, or 4:1, to 3:1, 2.5:1, or 2:1 of activated PBMC:stem or progenitor cells in said composition. In some embodiments, the activated PBMC and the stem or progenitor cells are present in a ratio of from 4.5:1, or 4:1, to 2.5:1, or 2:1 of activated PBMC:stem or progenitor cells in said composition.

In some embodiments, the stem or progenitor cells are placenta derived progenitor cells. In some embodiments, the stem or progenitor cells are mesenchymal stem cells.

In some embodiments, the activated PBMC are from a different subject than said stem or progenitor cells. In some embodiments, the activated PBMC and the stem or progenitor cells are both from the same subject.

In some embodiments, the activated PBMC are activated with antigens from a tissue selected from the group consisting of: cartilage, bone, muscle, nerve and brain tissue.

In some embodiments, the activated PBMC are activated with antigens from a tissue selected from the group consisting of: cartilage and bone tissue.

In some embodiments, the antigens are an enzymatic hydrolysate of the tissue.

In some embodiments, the composition is formulated for injection or infusion into an injured or diseased tissue.

In some embodiments, the activated PBMC and the stem or progenitor cells are co-cultured for less than 96, 72 or 48 hours prior to use (e.g., about 24 hours).

In some embodiments, the composition further comprises a hydrogel carrier and/or hyaluronic acid.

Also provided is a method of regenerating tissue in a subject in need thereof comprising administering a composition as taught herein to the subject in a treatment effective amount, or the use of a composition as taught herein for regenerating tissue in a subject in need thereof.

In some embodiments, the administration is carried out from about 4 or 5 days to about 11 or 12 days after tissue injury, such as about 6 days to about 10 days after tissue injury. In some embodiments, the administration is carried out about 1, 2, 3 or 4 weeks after tissue injury.

In some embodiments, the activated PBMC are obtained by extraction from whole blood, apheresis or buffy coat of the subject or a donor.

In some embodiments, the PBMC are autologous with respect to the subject in need thereof, and the stem or progenitor cells are allogenic with respect to the subject in need thereof.

In some embodiments, the composition is administered locally to the site of injury or disease of the subject in need thereof. In some embodiments, the site of injury or disease is the knee of the subject.

In some embodiments, the composition is administered systemically, and the PBMC and/or stem or progenitor cells migrate to the site of injury or disease of the subject in need thereof. In some embodiments, the site of injury or disease is the knee of the subject.

Further provided is a method of forming a tissue in vitro, comprising: providing a tissue scaffold; and seeding stem or progenitor cells and activated PBMC onto the scaffold, whereby the stem or progenitor cells differentiate into the tissue in vitro.

In some embodiments, the activated PBMC are activated with antigens from a tissue selected from the group consisting of: cartilage, bone, muscle, nerve and brain tissue.

In some embodiments, the stem or progenitor cells and activated PBMC are seeded onto the tissue scaffold together as a co-culture.

In some embodiments, the stem or progenitor cells and activated PBMC are seeded onto the tissue scaffold separately.

### BRIEF DESCRIPTION OF THE DRAWINGS

FIG. 1. In vitro mechanistic evaluation of the cartilage-activated effector cells (ECs) confirmed stimulation of macrophages and lymphocytes as shown by an increase in CD68, CD69 and CD25, as compared to the primary peripheral blood mononuclear cell population (MNC).

FIG. 2. Measurement of gene expression of the cartilage-activated ECs showed an increased expression of pro-regenerative markers TNF alpha and IL10 together with an increase in factors inducing chondrogenic differentiation, COMP and BMP2, as compared to the primary MNC population.

FIG. 3. Upon 24 hours of co-culture of the ECs with placenta derived progenitor cells (PLCs), the onset of chondrogenic differentiation was confirmed by the temporal upregulation of the transcription factors, cartilage matrix protein and the synovial lubricant Prg4.

FIG. 4. When the cell populations were injected in three models of dysfunctional healing, followed by pathological assessment 12 weeks post defect creation, the co-cultured cell treatment induced functional regeneration, whereas the other cell treatments with each of the cell populations alone did not. This was confirmed by the restoration of: (1) the tidemark between the subchondral bone and the articular unit, (2) subchondral articular cartilage rich in glycosaminoglycans, and (3) an articular chondrocyte layer secreting Lubricin, a qualitative marker for functional articular cartilage.

FIG. 5. Although the administration of the placenta cells alone produced anti-inflammatory effects and partial regeneration, it did not induce rapid tissue repair and functional regeneration like the co-cultured composition.

FIG. 6. Significant improvements in pain, functionality and quality of life were reported at 180 days post treatment in patients participating in a compassionate use study.

### DETAILED DESCRIPTION

The present invention is now described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete and will fully convey the scope of the invention to those skilled in the art.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

All publications, patent applications, patents and other references mentioned herein are incorporated by reference to the extent they are consistent with the disclosure set forth herein. In case of a conflict in terminology, the present specification is controlling.

As used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. Furthermore, a range provided herein for a measurable value may include any other range and/or individual value therein.

“Cells” used in the present invention are, in general, animal cells, particularly mammalian and primate cells, examples of which include, but are not limited to, human, dog, cat, rabbit, monkey, chimpanzee, cow, pig, or goat. The cells may be stem or progenitor cells, or differentiated at least in part to a particular cell or tissue type, such as cartilage, bone, muscle (smooth muscle, skeletal muscle, cardiac muscle), nerve (central nerve, peripheral nerve), brain tissue, etc. Cells may be obtained from established cultures, donors, biopsy, or a combination thereof, as known in the art.

In some embodiments, cells may be culture expanded prior to use. “Expanding” as used herein refers to an increase in number of viable cells, and may be accomplished by, e.g., growing the cells through one or more cell cycles, wherein at least a portion of the cells divide to produce additional cells.

“Subjects” as used herein are, in general, human subjects, although aspects of the invention may be implemented with other animal subjects, particularly mammalian subjects (e.g., dogs, cats, horses, goats, sheep) for research or veterinary purposes. Subjects may be male or female and of any age, including infant, juvenile, adolescent, adult, and geriatric.

“Treat” as used herein refers to any type of treatment that imparts a benefit to a subject, including, but not limited to, delaying the onset or reducing the severity of at least one symptom associated with tissue injury or disease such as osteoarthritis, improvement of tissue or joint function, etc. Similarly, a “treatment effective” amount is an amount that is sufficient to treat and provide such a benefit.

“Media” as used herein may be any natural or artificial growth media (typically an aqueous liquid) that sustains the cells used in carrying out the present invention. Examples include, but are not limited to, an essential media or minimal essential media (MEM), or variations thereof such as Eagle's minimal essential medium (EMEM) and Dulbecco's modified Eagle medium (DMEM). In some embodiments, the growth media includes a pH color indicator (e.g., phenol red).

As known in the art, stem and progenitor cells are cells that are able to differentiate into one or more specialized types of tissue cells. Stem cells generally can differentiate into a greater number of different types of cells than progenitor cells and can be culture expanded indefinitely. Either can be used in the present invention provided that they can be differentiated into cells of the tissue of interest, such as cartilage, bone, muscle, nerve or brain tissue. Stem or progenitor cells useful in the present invention include, but are not limited to, placenta derived progenitor cells, mesenchymal stem cells, periosteal stem cells, synovial membrane derived progenitor cells, meniscus derived progenitor cells, etc.

“Mesenchymal cells” or “mesenchymal stem cells” (“MSC”) are stem cells that can differentiate into a variety of cell types, such as osteoblasts, chondrocytes, myocytes, and adipocytes. In some embodiments, the MSC are from bone marrow. In some embodiments, the MCS are from adipose tissue.

“Placenta derived progenitor cells” or “PLCs” are progenitor cells that originate from a postpartum placenta and can be collected with methods known in the art. See, e.g., US 2008/0131410 and US 2014/0349391 to Hariri, and WO 2005/038012 A3 to Ethicon Incorporated, which are incorporated by reference herein. In some embodiments, the use of placenta derived progenitor cells is preferred due their favorable properties of immune system acceptance, availability, and expansion capability. As disclosed herein, placenta derived progenitor cells have been shown to be effective in the present invention, despite reports from prior studies that these cells have shown limited potential for chondrogenic differentiation in comparison to more evident cell sources such as bone marrow derived MSCs. See, e.g., Jeon et al., “Comparative Analysis of Human Mesenchymal Stem Cells Derived From Bone Marrow, Placenta, and Adipose Tissue as Sourced of Cell Therapy,” J. Cell Biol. 117 (5): 1112-1125 (2016).

“Peripheral blood mononuclear cells” (“PBMC” or “PBMNC”) are white blood cells with round nuclei and include a mixture of immune cells such as lymphocytes (T cells, B cells, NK cells), dendritic cells, and monocytes. PBMC may be obtained by art known methods such as by density gradient centrifugation of whole blood or blood apheresis, or a buffy coat (leukocyte concentrate).

PBMC may be “activated” by incubation with a tissue-specific antigen to activate the immune cells against that tissue. In some embodiments, PBMC may be activated by incubating the PBMC for about 1, 2, 3 or 4 days in an activation media containing a tissue antigen. In some embodiments, the tissue antigen is an enzymatic hydrolysate of a desired tissue (e.g., bone, cartilage, muscle, brain, or nerve tissue). In some embodiments, the activation media further includes sugar such as glucose, additive(s) such as one or more of a histamine type 2 receptor blocker (H2RAs or H2 antagonists, e.g., famotidine), a COX 1/2 inhibitor (e.g., indomethacin), an antibiotic, etc.

Activated PBMC (also called activated MC, or effector cells (EC) herein) may be confirmed by detection of stimulation of macrophages and lymphocytes, for instance, by detection of an increase in expression of markers CD68 and CD25, and in some embodiments CD69, and/or an increase in expression of pro-regenerative markers and/or factors relevant to induce differentiation into the tissue of interest (e.g., COMP and BMP2 for chondrogenic differentiation).

### Cell Compositions and Methods of Use

Compositions are provided which include activated PBMC and stem/progenitor cells in a co-culture. The PBMC are activated with antigens that correspond to a tissue to be regenerated (“tissue of interest”), and during an in vitro co-culture of the activated PBMC and stem/progenitor cells, the stem/progenitor cells are influenced to start down a path of differentiation to the specific tissue. This influence during the co-culture results in unexpectedly superior results in tissue regeneration upon administration of the co-culture to the tissue, with functional recovery and in some embodiments a fast (e.g., less than one week) tissue regeneration and functional recovery.

To provide an appropriate crosstalk of the cells, the activated PBMC and stem/progenitor cells such as placenta derived progenitor cells are provided in a ratio of from 6:1, 5:1, 4.5:1, or 4:1, to 3:1, 2.5:1, or 2:1 of PBMC:stem/progenitor cells in said composition. In some embodiments, the ratio is 4.5:1 to 2:1. In some embodiments, the ratio is about 4:1. In some embodiments, the ratio is about 3:1. While it is beneficial to have the activated PBMC to provide a synergistic effect in the crosstalk of the cells, too high of a ratio of the activated PBMC may result in significant apoptosis.

Administration of the cells may be by direct administration into the tissue, e.g., by injection of infusion, or may be systemic administration, where the cells (PBMC and/or stem or progenitor cells) will migrate to the site of injury or disease. For instance, in the case of cartilage regeneration, the cell co-culture may be injected or infused into the site of injury, such as an osteoarthritic knee, whereby the administration (e.g., intra-articular injection) results in functional regeneration of the articular cartilage of the knee, in some embodiments in a time of less than one week, less than two weeks, less than one month, less than two months, or less than three months.

In some embodiments, the co-culture composition also includes a hydrogel carrier (e.g., comprising collagen, gelatin, fibrin, combinations thereof, etc.) and/or hyaluronic acid.

The co-culture may also be used in a tissue engineering approach, such as by seeding the cells onto a tissue scaffold such as a three-dimensional matrix or hydrogel, with the co-culture activated towards a tissue of interest to be engineered. Although in some embodiments the activated PBMC and stem/progenitor cells may be seeded together onto the scaffold after co-culture, in other embodiments the stem/progenitor cells may be seeded first, followed by seeding of the activated PBMC.

“Scaffolds” on which cells may be seeded and grown to produce cultured tissue include any suitable support and/or three-dimensional matrix. See, e.g., U.S. Pat. Nos. 6,998,418; 6,485,723; 6,206,931; 6,051,750; and 5,573,784. Preferably, the scaffold is configured to support the attachment, proliferation and/or differentiation of cells thereon. The scaffold may be formed from any suitable material, including, but not limited to, synthetic or natural polymers, other biopolymers, and combinations thereof. In some embodiments, scaffolds include collagen supports or decellularized tissue supports. In some embodiments, the scaffold may include an electrospun matrix. In some embodiments, scaffolds include a polymeric matrix (e.g., collagen, a hydrogel, etc.). In some embodiments, scaffolds include fibrin or fibrinogen.

The present invention is explained in greater detail in the following non-limiting examples.

### EXAMPLES

### Example 1: Functional Regeneration of a Severely Damaged Osteochondral Unit

We have developed an immunomodulatory cell treatment that can be injected intra-articular (IA) in the early phase of PTOA initiation or during disease progression, and has shown significant promise as a therapeutic in a small animal model of PTOA. Our breakthrough treatment is based on a combination cell product that has shown efficacy when given IA in experimentally injured rats.

Injury to the knee leads to an immediate activation of pro-inflammatory neutrophils, which decontaminate and clear the site and guide infiltrating monocytes. Secreted cytokines at the wound site steer differentiation of the monocytes into macrophages, which remove apoptotic cells and function as antigen-presenting cells specific for the injured tissue. This step prevents further leukocyte influx and steers the injured environment towards a pro-regenerative process. In functional regeneration, the completion of the proinflammatory phase leads to recruitment of the pro-regenerative immune cells, and in turn progenitor cell recruitment, a crucial step for functional healing to occur. The signaling cascades that steer the balance, polarization and subsequent action of the recruited immune cells, including T helper (Th) 1, Th2 and Th17 and T regulatory (Treg) cells, macrophages and mast cells, are crucial for functional transition. Therefore, we hypothesized that failed regeneration of cartilage may be caused by an imbalance between the pro-inflammatory and pro-regenerative cells that results in a prolonged inflammation and fibrosis.

In an effort to study and discover interventions in PTOA, we developed four rat models of osteochondral defect (OCD) and subsequent healing. These four separate models represent different levels of insult and regeneration. For this, full thickness OCD with small diameter (SD) and large diameter (LD) were created in the trochlear groove of 10-week-old wild type (WT) and mature T cell deficient (T cell-) female rats, and followed for 12 weeks. Tissue healing, progenitor cell activation and extracellular matrix (ECM) production at 96 h, and 1, 4 and 12-weeks post defect creation were assessed. Functional healing was confirmed in SD-WT animals, while different degrees of dysfunctional healing and fibrosis was observed in WT-LD, T cell-SD and -LD joints. These experiments displayed a direct correlation between the inflammatory reaction and progenitor cell activation at one week post injury, within each model's healing status.

Based on these findings, a cell-based treatment was developed with the aim to facilitate the pro-regenerative processes. For comparison, three specific cell populations were developed: (1) rat mononuclear cells, isolated and activated with a cartilage antigen to induce a cartilage-activated effector cell (EC) population; (2) in vitro expanded rat placenta derived progenitor cells (PLCs); and (3) a 24 h co-cultured combination of ECs and PLCs in a 4:1 ratio.

EC can be obtained from Peripheral Blood Mononuclear Cells (PBMNC, or MNC), extracted from total blood, or apheresis of the patient or a donor. The PBMNC are purified and obtained through a gradient of Ficoll-Hypaque. PBMNC were activated during a 72 hr period in DMEM containing 4500.0 mg/ml Glucose and GlutaMAx, with 10% hydrolyzed antigen (cartilage), 10 μl/ml Famotidine and 4.5 μl/ml Indomethacin. PLCs were obtained from the Wake Forest Institute for Regenerative Medicine's clinical manufacturing center and expanded in growth media. The co-cultured cell product is obtained through the co-incubation of EC with PLCs at a 4:1 ratio for 24 h in serum, and antigen-free media.

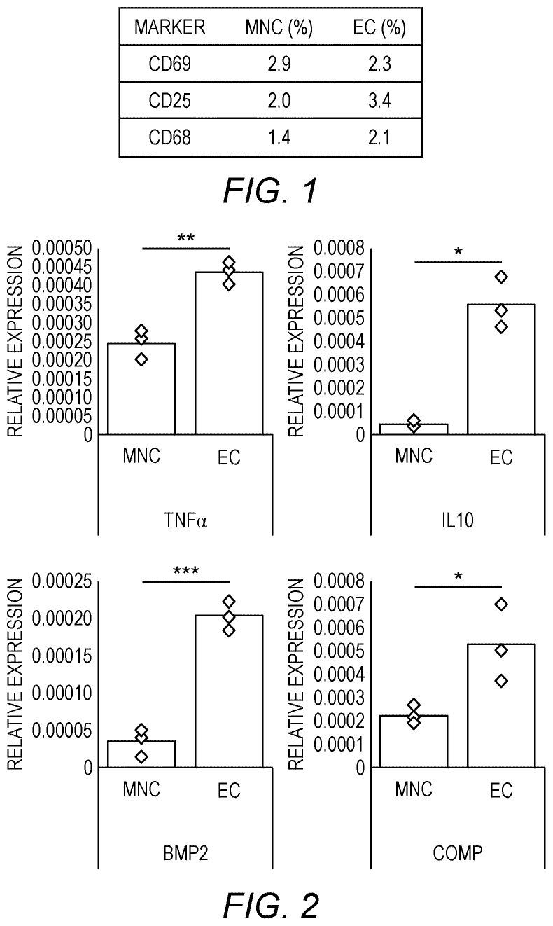

In vitro mechanistic evaluation of the cartilage-activated ECs confirmed stimulation of macrophages and lymphocytes as shown by an increase in CD68, CD69 and CD25, FIG. 1, as compared to the primary PBMNC population. These findings were further supported by gene expression, where an increased expression of pro-regenerative markers TNF alpha and IL10 were observed together with an increase in factors inducing chondrogenic differentiation, COMP and BMP2, FIG. 2.

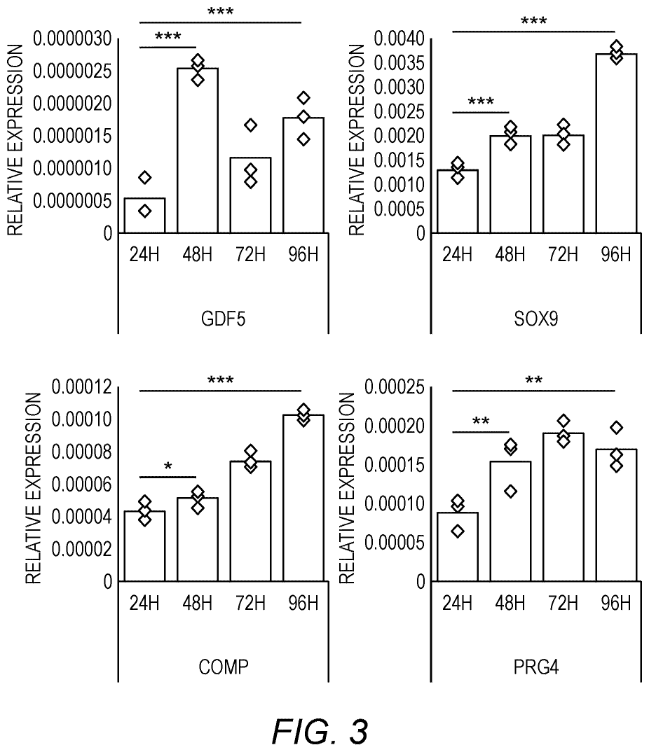

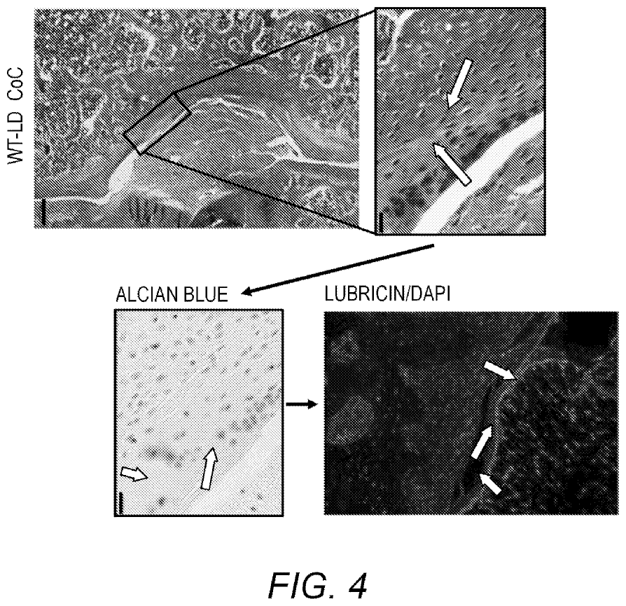

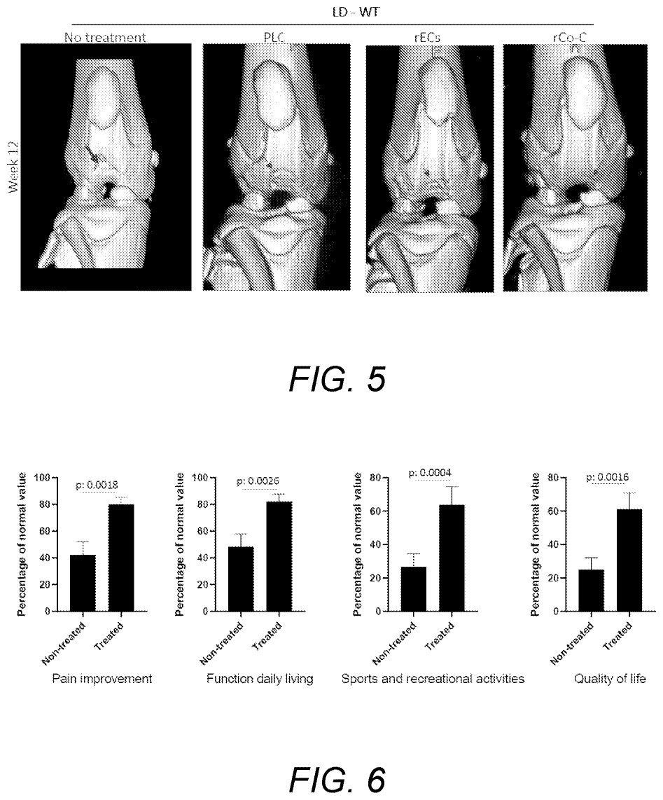

Upon 24 hr of co-culture of the ECs with PLCs, the onset of chondrogenic differentiation was confirmed by the temporal upregulation of the transcription factors, cartilage matrix protein and the synovial lubricant Prg4, FIG. 3. When these cell populations were injected in the three models of dysfunctional healing, followed by pathological assessment 12 weeks post defect creation, the co-cultured cell treatment induced functional regeneration, FIG. 4, whereas the other cell treatments with each of the cell populations alone did not. This was confirmed by the restoration of: (1) the tidemark between the subchondral bone and the articular unit, (2) subchondral articular cartilage rich in glycosaminoglycans, and (3) an articular chondrocyte layer secreting Lubricin, a qualitative marker for functional articular cartilage. Although the administration of the placenta cells alone produced anti-inflammatory effects and partial regeneration, it did not induce rapid tissue repair and functional regeneration like the co-cultured composition, FIG. 5.

These results demonstrate for the first time that functional regeneration of a severely damaged osteochondral unit can be achieved with an injectable immune modulatory cell-based treatment, a goal of the entire field for over four decades.

### Example 2: Repeated Injections of the Cell-Based Treatment

CT-scans were performed on rat synovial joints: (A) after repeated injections (weeks 1 and 3) of the co-cultured cartilage activated mononuclear cells (ECs) and placenta derived progenitor cells (PLCs), (B) with a delayed injection two weeks post OCD creation, and (C) with a delayed treatment four weeks post OCD creation. The results indicated that repeated treatment does not induce an expansion of the subchondral bone or osteophytes, while the delayed treatment at two weeks shows satisfactory regeneration of the mineralized tissue, and the four-week injection shows some remaining defect.

These data suggest that approximately one week post injury may be the most favorable time for administration, although later time points are still showing significant improvement in healing.

### Example 3: Nanostring Analysis of Six Donor Populations

Mononuclear cells from six different patients were activated to become cartilage activated effector cells, and nanostring analysis was performed to evaluate the activation into specific T-cell populations. The resulting heatmap confirmed that MNC from different donors respond very similar, confirming that limited donor-variability is expected upon co-culture. The nanostring analysis further confirmed that the activation of the six donor populations led to a reduced expression of pro-inflammatory cytokines IL17A, IL17C and IL17F.

In addition, the nanostring analysis further confirmed that the activation of the six donor populations and the subsequent induction of chondrogenic differentiation is related to activated BMP-signaling, indicated by elevated SMAD2 and SMAD 3 expression. SQSTM is a gene related to tissue remodeling. Specifically, it is an autophagosome cargo protein that targets other proteins that bind to it for selective autophagy by interacting with GATA4 and targeting it for degradation, it can inhibit GATA-4 associated senescence and senescence-associated secretory phenotype, and it is also involved in recycling of worn-out cell parts and unneeded proteins, the self-destruction of cells (apoptosis), and the body's immune responses and inflammatory reactions. Based on the upregulation, and without wishing to be bound by theory, this may be a key factor underlying the potent pro-regenerative effect seen in the ECs.

### Example 4: Compassionate Use Study in Human Patients

Nine patients (6 male, 3 female) were enrolled in a compassionate use study and received injections of the cell-based treatment, with use of autologous adipose-derived cells as stem/progenitor cells. Patients offered the treatment were at least 18 years of age and had consulted the clinic for knee osteoarthritis (KOA), with a body weight greater than 45 Kg and blood parameters: Hematocrit≥35%; MCV≥70%; MCH≥31%; Leukocytes≥4000/mm³; Platelets: 150000 to 400000/mm³; with normal cardiac, liver, respiratory, and renal functions. Inclusion criteria: Positive KOA diagnosis supported by clinical symptoms and X-ray knee abnormalities and confirmed by MRI of the knee (effusion and synovial thickening/synovitis, subchondral bone marrow edema and/or cysts, cartilaginous defects (partial or full-thickness) bursitis, iliotibial band syndrome). The pain associated with the MRI characteristics, according to the KOOS scale, was higher than 80%. Exclusion Criteria: Active neoplasm, viral, bacterial or fungal (internal) active systemic infection at the time of starting the treatment, HIV, hepatitis B or hepatitis C positive, patients with jaundice or liver failure, pregnant patients, or individuals with alcohol or drug addiction.

All included patients suffered from severe KOA. Their indication was prosthetic replacement of the knee as OA progression caused a lack of treatment response to standard palliative therapies. The treatment was done under Compassionate Use Conditions. Patient safety and clinical follow-up was monitored at least once a week to observe treatment safety, efficacy, and effectiveness. Follow up controls were done at 60, 90, and 180 days, and data from day 180 was used for this study. Safety was evaluated according to the National Cancer Institute. USA. Common terminology criteria for adverse events (version 5.0), Koos Index was assessed at 3 months. MRI of the treated areas was performed 6 months after the end of the treatment. Statistical analysis of the clinical data was done with the MedCalc Software from MedCalc Inc.

As shown in FIG. 6, there were significant improvements in pain, functionality and quality of life reported by patients at 180 days post treatment.

The foregoing is illustrative of the present invention, and is not to be construed as limiting thereof. The invention is defined by the following claims, with equivalents of the claims to be included therein.

1. A composition comprising a co-culture of:
a) activated peripheral blood mononuclear cells (PBMC); and
b) stem or progenitor cells,
wherein said activated PBMC and said stem or progenitor cells are present in a ratio of from 6:1, 5:1, 4.5:1, or 4:1, to 3:1, 2.5:1, or 2:1 of activated PBMC:stem or progenitor cells in said composition.

2. The composition of claim 1, wherein said stem or progenitor cells are placenta derived progenitor cells.

3. The composition of claim 1, wherein said stem or progenitor cells are mesenchymal stem cells.

4. The composition of claim 1, wherein said activated PBMC are from a different subject than said stem or progenitor cells.

5. The composition of claim 1 wherein said activated PBMC and said stem or progenitor cells are both from the same subject.

6. The composition of claim 1, wherein said activated PBMC are activated with antigens from a tissue selected from the group consisting of: cartilage, bone, muscle, nerve and brain tissue.

7. The composition of claim 1, wherein said activated PBMC are activated with antigens from a tissue selected from the group consisting of: cartilage and bone tissue.

8. The composition of claim 6, wherein the antigens are an enzymatic hydrolysate of the tissue.

9. The composition of claim 1, wherein said composition is formulated for injection or infusion into an injured or diseased tissue.

10. The composition of claim 1, wherein the activated PBMC and the stem or progenitor cells are co-cultured for less than 96, 72 or 48 hours prior to use (e.g., about 24 hours).

11. The composition of claim 1, further comprising a hydrogel carrier and/or hyaluronic acid.

12. A method of regenerating tissue in a subject in need thereof comprising administering the composition of claim 1 to the subject in a treatment effective amount.

13. The method of claim 12, wherein the activated PBMC are obtained by extraction from whole blood, apheresis or buffy coat of the subject or a donor.

14. The method of claim 12, wherein the PBMC are autologous with respect to the subject in need thereof, and the stem or progenitor cells are allogenic with respect to the subject in need thereof.

15. The method of claim 12, wherein the composition is administered locally to the site of injury or disease of the subject in need thereof.

16. The method of claim 15, wherein the site of injury or disease is the knee of the subject.

17. The method of claim 12, wherein the composition is administered systemically, and the PBMC and/or stem or progenitor cells migrate to the site of injury or disease of the subject in need thereof.

18. The method of claim 17, wherein the site of injury or disease is the knee of the subject.

19. A method of forming a tissue in vitro, comprising:
providing a tissue scaffold; and
seeding stem or progenitor cells and activated PBMC onto the scaffold,
whereby the stem or progenitor cells differentiate into the tissue in vitro.

20. The method of claim 19, wherein the activated PBMC are activated with antigens from a tissue selected from the group consisting of: cartilage, bone, muscle, nerve and brain tissue.

21. The method of claim 19, wherein the stem or progenitor cells and activated PBMC are seeded onto the tissue scaffold together as a co-culture.

22. The method of claim 19, wherein the stem or progenitor cells and activated PBMC are seeded onto the tissue scaffold separately.
